(19) United States
(12) Patent Application Publication
(10) Pub. No.: US 2006/0025724 A1
(43) Pub. Date: Feb. 2, 2006

(54) Medical connector with valve

(75) Inventor: Wei Chen, Houston, TX (US)

Correspondence Address: Richard L. Moseley, P.O. Box 630708, Houston, TX, 77263, US

(73) Assignee: GLOBE MEDICAL TECH, INC.

(21) Appl. No.: 10/899,108

(22) Filed: Jul. 27, 2004

(51) Int. Cl. A61M 5/00

(52) U.S. Cl. 604/249; 251/149.1

(57) ABSTRACT

A needleless connector for adding medication to a parenteral fluid is provided. The connector is provided with a male luer connector at one end and a female connector at the opposite end. A sliding plug valve is built into the connector so that the parental fluid may be isolated from the injection site to prevent back flow when the syringe is removed.

### BACKGROUND OF THE INVENTION

1. Field of the Invention

This invention relates to medical connectors used in the introduction of liquids into patients via intravenous solutions. More particularly the invention relates to a needleless connection for use with syringes or other apparatus having a standard luer lock type connection.

2. Related Information

It is a common practice in treating patients, particularly patients who must be cared for under emergency conditions, to use medications introduced into the patient intravenously. An intravenous solution, commonly referred to as parenteral fluid, is fed from a container or I.V. bag through a tubing and a catheter which has been inserted into the patient's vein. The catheter is secured to the patient by a strip of adhesive tape The medication to be administered is generally added to the parenteral fluid through a Y connector in the tubing. The conventional practice has been to inject the fluid using a hypodermic syringe and needle through a sealed entry port in the Y connector in the tubing. Generally the seal at the port is a puncture pad of generic latex which for a limited number of uses will reseal itself when a needle is withdrawn.

One problem with this conventional practice is that the needle may be pulled loose from the sealed port very easily. Another problem is needle sticks. From time to time a nurse in attempting to insert the needle into the port will accidentally stick himself or herself with the needle. While there is little risk of infection to the nurse if the needle is new and sterile, the process may be slowed until the needle has been replaced. Lopez, et al in U.S. Pat. No. 4,752,292 have presented one solution to the problem.

The connector of Lopez, et al presumes that the needle connector may be easily attached to the source of medication. When measured doses are necessary syringes are more often used and the Lopez connector is not practical.

Another problem associated with injecting fluids is the possibility of back flow when the syringe is removed.

It is an object of the present invention to provide a needleless connector for administering medication through a Y connector of an I.V. system that prevents back flow.

### SUMMARY OF THE INVENTION

In its simplest form the present invention comprises a connector adapted to receive a syringe and having a valve to seal the port in place of a puncture pad. The valve comprises a housing having a port that is sealed by a sliding plug. The sliding plug has an aperture therethrough that when aligned with the port allows liquid to flow through the valve. At one end the housing has a male luer connection and at the opposite end it has a female luer connector. The male luer connector includes a rubber spring type bladder valve that is actuated by insertion of the luer connection of a syringe. The plug includes O rings on either side of the aperture and also around the edge of the aperture.

### BRIEF DESCRIPTION OF THE DRAWING

FIG. 1 is an exploded plan view of the connector of the present invention showing the individual parts.

FIG. 2 is a plan view in cross section of the connector of the present invention.

FIG. 3 is a side view of the assembled connector of the present invention.

### DESCRIPTION OF THE PREFERRED EMBODIMENT

For a detailed description of the preferred embodiment the reader is directed to the accompanying figures in which like components are given like numerals for ease of reference.

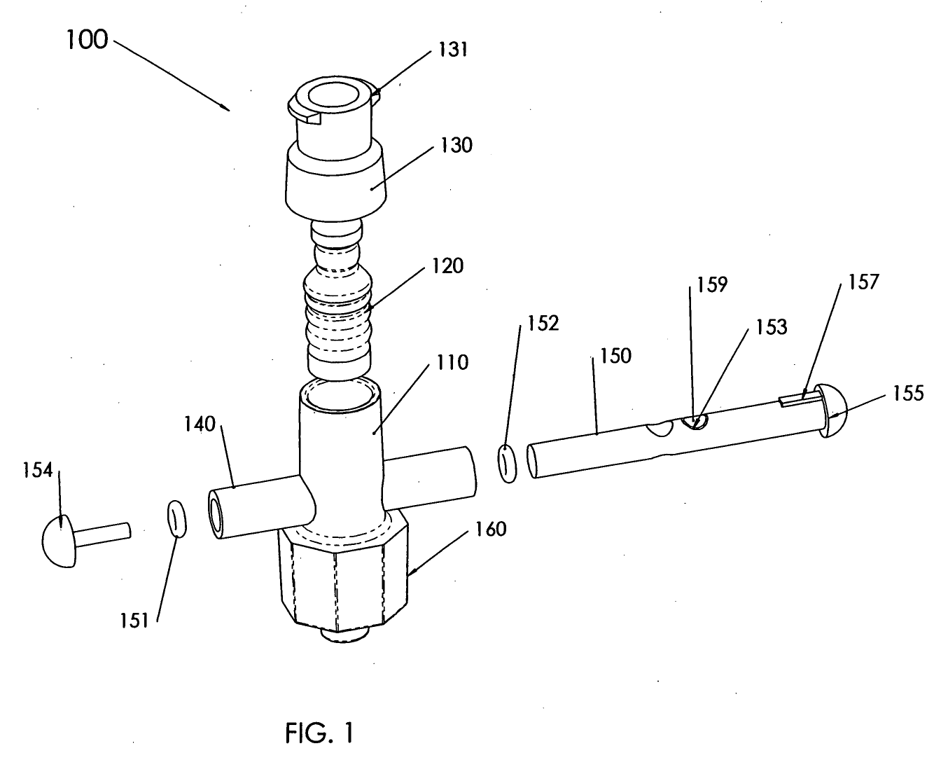

Referring first to FIG. 1 there is shown an exploded view of the connector 100 of the present invention. The connector is seen to comprise a housing 110 into which is inserted from the top end a rubber spring bladder 120 which is held in place by a lock top 130 which includes a male luer connection 131 at the upper end.

In approximately the center of the housing 110 is a slide receiver 140 for receiving a sliding plug 150. The interior of the slide receiver 140 is a hollow cylinder while the sliding plug 150 is itself is a solid cylinder having an aperture 159 therethrough. The inner surface of the slide receiver contains a positioning channel 156 for receiving a positioning key 157 which is located on the outer surface at one end of the sliding plug 150. The positioning key 157 prevents the sliding plug 150 from rotating in the receiver 140. The O rings 151 and 152 fit about the sliding plug between the ends and the aperture 159. Another O ring 153 surrounds the aperture 159. End caps 154 and 155 are placed at either end of the sliding plug 150. The lower end of the housing 110 comprises a male luer connection 160.

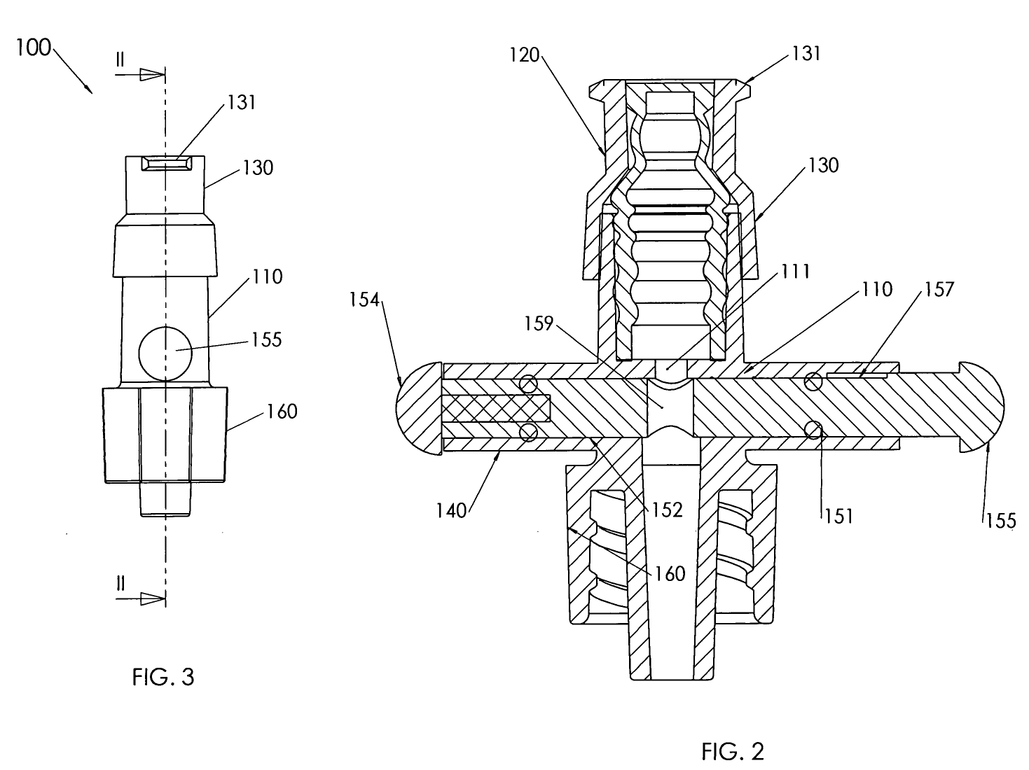

Referring now to FIGS. 2 and 3 the assembled connector 100 is shown. The sliding plug 140 is shown in the open position with the aperture 159 aligned with the port 111 in the housing. Aperture O ring 153 seals around the port 111 to prevent leakage around the plug. When the slide plug is moved away from center the aperture is no longer aligned with the port and the valve is sealed by the plug O rings 151 and 152. The aperture 159 is positioned in the sliding plug such that it is aligned with the port 111 when one of the caps (here 155) is snug against the end of the receiver 140. When the opposite cap 154 is snug against the receiver 140 the valve is in the closed position.

In use a syringe having a female luer connector (not shown) is attached to the male connector of the connector. This pushes the rubber spring bladder 120 downward and allows fluid to enter the housing. The slide plug 140 is moved into the open position and the fluid is injected into the IV system (not shown) which is connected to the lower end female luer connection 160. After injection is complete the sliding plug 140 is moved to the closed position to prevent back flow when the syringe is removed.

The housing, slide receiver, slide and luer connectors are preferably made of clear hard plastic. The O rings and rubber bladder are preferably made of medical grade rubber.

It will be apparent to those skilled in the art that many modifications and changes in the apparatus may be made without departing from the scope and spirit of the invention. It is the intention, therefore, that the following claims cover all equivalent modifications and variations and fall within the scope of the invention as defined by the claims.

The invention claimed is:

1. A medical connector comprising;
a housing having a male luer connection at one end and a female luer connection at the opposite end;
a slide receiver intermediate said ends;
a rubber spring bladder disposed within said housing proximate said male luer connector;
a port in said housing immediately above said slide receiver; and
a sliding plug disposed within said receiver having an aperture therethrough alignable with said port.

2. The medical connector of claim 1 wherein said slide receiver is a hollow cylinder and said sliding plug is a solid cylinder having an aperture therethrough alignable with said port.

3. The medical connector of claim 2 further comprising a positioning channel in the inner surface of said slide receiver and a positioning key on the upper surface of said sliding plug.

4. The medical connector of claim 3 further comprising an aperture O ring disposed about said aperture adjacent said port.

5. The medical connector of claim 3 further comprising two plug O rings, one each of said plug O rings disposed about said sliding plug between the aperture and each end.

6. A medical connector comprising;
a housing having a male luer connection at one end and a female luer connection at the opposite end;
a hollow cylindrical slide receiver intermediate said ends;
a rubber spring bladder disposed within said housing proximate said male luer connector;
a port in said housing immediately above said slide receiver;
a cylindrical sliding plug disposed within said receiver having an aperture therethrough alignable with said port;
an aperture O ring disposed about said aperture adjacent said port; and
two plug O rings, one each of said plug O rings disposed about said sliding plug between the aperture and each end.
